(19) United States
(12) Patent Application Publication
(10) Pub. No.: US 2025/0325554 A1
(43) Pub. Date: Oct. 23, 2025

(54) METHODS AND PHARMACEUTICAL COMPOSITIONS TO TREAT RESPIRATORY DEPRESSION IN PATIENTS

(71) Applicant: Enalare Therapeutics Inc., Princeton, NJ (US)

(72) Inventors: Joseph PERGOLIZZI, Princeton, NJ (US); Robert B. RAFFA, Princeton, NJ (US); Alfred SCHWEIKERT, Princeton, NJ (US)

(21) Appl. No.: 18/866,742

(22) PCT Filed: May 18, 2023
(86) PCT No.: PCT/US2023/022705

§ 371 Date: Nov. 18, 2024

Related U.S. Application Data
(60) Provisional application No. 63/343,744, filed on May 19, 2022.

(51) Int. Cl. A61K 31/5377; A61K 31/13; A61K 31/4706; A61K 31/505; A61K 31/519; A61K 31/53; A61K 31/7052; A61K 45/06

(52) U.S. Cl. CPC A61K 31/5377; A61K 31/13; A61K 31/4706; A61K 31/505; A61K 31/519; A61K 31/53; A61K 31/7052; A61K 45/06

(57) ABSTRACT

The present disclosure provides a method of treating respiratory depression in a patient after discontinuation of ventilator therapy comprising administering a respiratory stimulant to a patient in need thereof.

### BACKGROUND OF THE INVENTION

A mechanical ventilator, or ventilator, is a common medical device used to help push air in and out of the lungs so that the body can get oxygen it needs and eliminate carbon dioxide when the body cannot breathe properly on its own. There are a variety of reasons for which a person may be put on a ventilator, such as acute respiratory distress syndrome (ARDS), exacerbations of chronic obstructive pulmonary disease (COPD) or asthma, brain injury, cardiac arrest, pneumonia, a collapsed lung, a stroke, coma, drug overdose, hypercapnic respiratory failure, spinal cord injury, amyotrophic lateral sclerosis (ALS), a bacteria or viral infection or during surgery. When a patient has recovered enough, such that he or she can breathe on their own, the patient is weaned off of the ventilator.

It has been found that after prolonged time on a ventilator, the patient may suffer from demyelination, which may cause respiratory depression. Demyelination may be acute or chronic and may affect various parts of the nervous system, such as the central nervous system.

It has also been found that after prolonged time on a ventilator, the lungs may be stiffer, requiring a greater respiratory drive to achieve sufficient respiratory needs in a patient. It has also been found that some patients may experience atelectasis, or a decrease in lung compliance following periods of mechanical ventilation. In these instances, atelectasis and a decrease in lung compliance may require additional neural respiratory drive to achieve adequate spontaneous ventilation compared to neural effort required prior to the event. If the additional neural respiratory drive is not achieved, it can manifest as inadequate or depressed ventilation, such as respiratory depression.

There exists a need in the art for treatment of patients who have respiratory depression after being on a ventilator.

### Objects and Summary

In certain embodiments, the disclosure is directed to a method of treating respiratory depression in a patient after discontinuation of ventilator therapy comprising administering a respiratory stimulant to a patient in need thereof.

In other embodiments, the disclosure is directed to a pharmaceutical composition comprising an effective amount of a respiratory stimulant to treat respiratory depression in a patient after discontinuation of ventilator therapy.

In other embodiments, the disclosure is directed to a method of treating respiratory depression in a patient comprising administering a respiratory stimulant, wherein the respiratory depression is caused by demyelination.

In other embodiments, the disclosure is directed to a method of treating respiratory depression in a patient comprising administering a respiratory stimulant, wherein the respiratory depression is caused by demyelination (e.g., acute or chronic).

### DETAILED DESCRIPTION

As used herein, the term “respiratory depression” is understood by one of skill as a breathing disordered characterized by slow and ineffective breathing. Respiratory depression may also be known as hypoventilation or hypoventilatory syndrome. Respiratory depression is the abnormal retention of carbon dioxide in the blood due to poor exchange of carbon dioxide and oxygen within the lungs. The poor exchange may be caused by inadequate ventilatory drive or inadequate respiratory drive in a patient.

In certain embodiments, the present invention is directed to a method of treating respiratory depression in a patient after discontinuation of ventilator therapy comprising administering a respiratory stimulant to a patient in need thereof.

In certain embodiments, the ventilator therapy is to treat respiratory depression in a patient experiencing a bacterial or viral infection. In certain embodiments of the method, the respiratory depression after discontinuation of ventilator treatment is caused by demyelination. In certain embodiments of the method, the respiratory depression after discontinuation of ventilator treatment is caused by atelectasis and reduced lung compliance. In certain embodiments, the ventilator therapy is to treat respiratory depression caused by administration of an active agent.

In certain embodiments, the methods of the present invention further comprise administering hydroxychloroquine to the patient.

In certain embodiments, the methods of the present invention further comprise administering an antibiotic to the patient. The antibiotic can be, e.g., a penicillin, cephalosporin, or a macrolide. In certain embodiments, the antibiotic is azithromycin.

In certain embodiments, the methods of the present invention further comprise administering an antiviral to the patient, e.g., amantadine

In certain embodiments, the methods of the present invention further comprise administering a bronchodilator to the patient, e.g., albuterol.

In certain embodiments, the methods of the present invention further comprise administering a lung recruitment maneuver.

In certain embodiments, the respiratory depression is caused by demyelination after being on a ventilator for a prolonged time period, by atelectasis associated with periods of mechanical ventilation or by administration of an active agent to treat the underlying reason why a patient is on a ventilator, such as acute respiratory distress syndrome (ARDS), chronic obstructive pulmonary disease (COPD), asthma, brain injury, cardiac arrest, pneumonia, a collapsed lung, a stroke, coma, drug overdose, hypercapnic respiratory failure, spinal cord injury, amyotrophic lateral sclerosis (ALS), or a bacteria or viral infection or a symptom related or unrelated to the underlying cause. In certain embodiments, the active agent is to treat pain, cough or constipation such as an opioid analgesic.

In certain embodiments, the methods of the present invention triggers ventilation in the lungs, e.g., through a physiological mechanistic process such as through the carotid bodies.

In certain embodiments, the respiratory stimulant is selected from the group consisting of Doxapram, Almitrine, ENA-001 (formerly GAL-021), and a pharmaceutically-acceptable salt thereof.

In certain embodiments, the respiratory stimulant is at least one compound of formula (I):

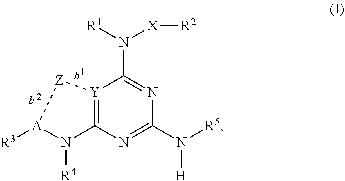

wherein:
R¹ and R² are independently H, alkyl, substituted alkyl, cycloalkyl, substituted cycloalkyl, alkenyl, substituted alkenyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, cycloalkyl, substituted cycloalkyl, alkenyl, substituted alkenyl, NR¹R², C(O)OR¹, acyl, or aryl;
R⁴ is H, alkyl, or substituted alkyl;
R⁵ is H, alkyl, substituted alkyl, cycloalkyl, substituted cycloalkyl, alkenyl, substituted alkenyl, −OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C; or a salt thereof.

In certain embodiments, the active agents are administered simultaneously or sequentially. In certain embodiments, at least two active agents are administered sequentially.

In certain embodiments, the active agents are administered by the same route of administration. In certain embodiments, the active agents are administered by at least two different routes of administration.

In certain embodiments, the active agents are administered in the same pharmaceutical composition or the active agents are administered in at least two different pharmaceutical compositions.

In certain embodiments, the active agent or agents are administered simultaneously or sequentially with a lung recruitment maneuver.

In certain embodiments, the stimulant is provided at a therapeutic dose which is sufficient to restore the respiratory rhythm of the patient.

In certain embodiments, the stimulant is provided at a therapeutic dose which does not induce hyperventilation or substantial hyperventilation in the patient.

In certain embodiments, the route is selected or the routes are independently selected from oral, intravenous, nasal, inhalational, topical, buccal, rectal, pleural, peritoneal, vaginal, intramuscular, subcutaneous, transdermal, epidural, intratrachael, otic, intraocular, or intrathecal route.

In certain embodiments, the infection can be coronavirus (e.g., Covid-19), SARS, MERS, swine flu or Zika virus.

In other embodiments, the present invention is directed to a method of treating respiratory depression in a patient comprising administering a respiratory stimulant, wherein the respiratory depression is caused by demyelination. In certain embodiments, the demyelination is caused by ventilator therapy.

In other embodiments, the present invention is directed to a method of treating respiratory depression in a patient comprising administering a respiratory stimulant, wherein the respiratory depression may be caused by atelectasis. In certain embodiments, the atelectasis may be caused by ventilator therapy.

In other embodiments, the present invention is directed to a pharmaceutical composition comprising an effective amount of a respiratory stimulant to treat respiratory depression in a patient after discontinuation of ventilator therapy and at least one of a chloroquine such as hydroxychloroquine, an antibiotic or an antiviral as disclosed herein.

In certain embodiments of the pharmaceutical composition, the ventilator therapy is to treat respiratory depression in a patient experiencing a bacterial or viral infection. In certain embodiments, the respiratory depression after discontinuation of ventilator treatment is caused by demyelination. In certain embodiments, the respiratory depression after discontinuation of ventilator treatment is caused by atelectasis. In certain embodiments of the pharmaceutical composition, the antibiotic is azithromycin.

In certain embodiments of the pharmaceutical composition, the antiviral is amantadine.

In certain embodiments of the pharmaceutical composition, the respiratory stimulant is selected from the group consisting of Doxapram, Almitrine, ENA-001, and a pharmaceutically-acceptable salt thereof.

In certain embodiments of the pharmaceutical composition, the respiratory stimulant is at least one compound of formula (I):

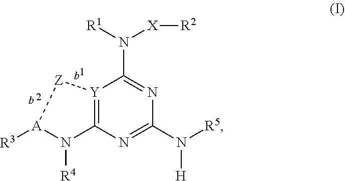

wherein:
R¹ and R² are independently H, alkyl, substituted alkyl, cycloalkyl, substituted cycloalkyl, alkenyl, substituted alkenyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, cycloalkyl, substituted cycloalkyl, alkenyl, substituted alkenyl, NR¹R², —C(O)OR¹, acyl, or aryl;
R⁴ is H, alkyl, or substituted alkyl;
R⁵ is H, alkyl, substituted alkyl, cycloalkyl, substituted cycloalkyl, alkenyl, substituted alkenyl, —OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C; or a salt thereof.

### Other Compounds that can be Used in Certain Embodiments

In other embodiments, the compound is of formula (I) or a salt thereof:

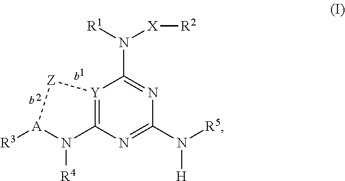

wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl or substituted alkynyl;
R⁴ is H, alkyl, or substituted alkyl;
R⁵ is alkyl, propargylic, substituted propargylic, homopropargylic, or substituted homopropargylic,
wherein at least one substituent selected from the group consisting of R¹, R², R³ and R⁵ is alkynyl or substituted alkynyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and:
(i) Z is H, bond b² is a single bond, and A is CH; or,
(ii) Z is nil, bond b² is nil, and A is a single bond; and,
if Y is C, then bond b¹ is a single bond, and:
(i) Z is CH₂, bond b² is a single bond, and A is CH; or,
(ii) Z is CH, bond b² is a double bond, and A is C.

In certain embodiments, (i) R³ is H, alkyl or substituted alkyl, and R⁵ is propargylic, substituted propargylic, homopropargylic, or substituted homopropargylic, or (ii) R³ is H or alkynyl, and R⁵ is alkyl, propargylic, substituted propargylic, homopropargylic, or substituted homopropargylic.

In certain embodiments, the compound is at least one selected from the group consisting of:

(i) Y is N, bond b¹ is nil, Z is H, bond b² is a single bond, A is CH, and the at least one compound is a compound of formula (II-a):

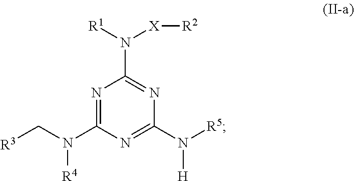

(ii) is N, bond b¹ is nil, Z is nil, bond b² is nil, and A is a bond, and the compound of the invention is a 1,3,5-triazine of formula (II-b);

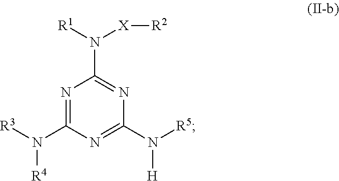

(iii) Y is CR⁶, bond b¹ is nil, Z is H, bond b² is a single bond, A is CH, and the at least one compound is a compound of formula (III-a);

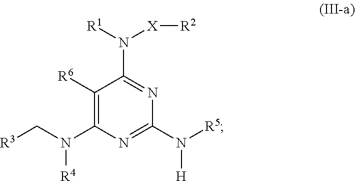

(iv) Y is CR⁶, bond b¹ is nil, Z is nil, bond b² is nil, and A is a bond, and the compound of the invention is a pyrimidine of formula (III-b):

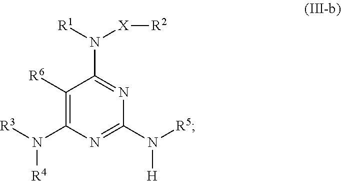

(v) Y is C, bond b¹ is a single bond, Z is CH₂, bond bis a single bond, A is CH, and the at least one compound is a compound of formula (IV):

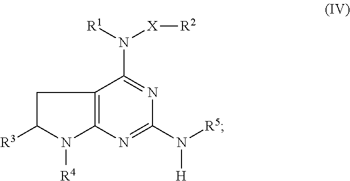

(vi) Y is C, bond b¹ is a single bond, Z is CH, bond b² is a double bond, A is C, and the at least one compound is a compound of formula (V):

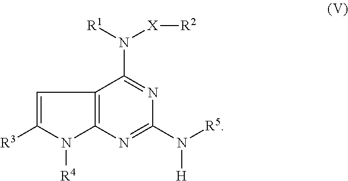

In certain embodiments, the compound is selected from the group consisting of:
O,N-Dimethyl-N-[4(-n-propylamino)-6-(prop-2-ynylamino)-[1,3,5]triazin-2-yl]-hydroxylamine;
N-Methyl-N′-n-propyl-N″-prop-2-ynyl-[1,3,5]triazine-2,4,6-triamine;
N-(4-Fluorobenzyl)-O-methyl-N-[4-(n-propylamino)-6-(prop-2-ynylamino)-[1,3,5]triazin-2-yl]-hydroxylamine;
N-(4-Fluorobenzyl)-N′-n-propyl-N″-prop-2-ynyl-[1,3,5]triazine-2,4,6-triamine;
N-[4-(4-Fluorobenzylamino)-6-(prop-2-ynylamino)-[1,3,5]triazin-2-yl]-O,N-dimethyl-hydroxylamine;
N-(4-Fluoro-benzyl)-N-[4-(4-fluorobenzylamino)-6-(prop-2-ynylamino)-[1,3,5]triazin-2-yl]-O-methyl-hydroxylamine;
N,N′-Bis-(4-fluorobenzyl)-N″-prop-2-ynyl-[1,3,5]triazine-2,4,6-triamine;
N-(4,6-Bis-prop-2-ynylamino-[1,3,5]triazin-2-yl)-O,N-dimethyl-hydroxylamine;
N-Methyl-N′,N″-di-prop-2-ynyl-[1,3,5]triazine-2,4,6-triamine;
N,N′-Bis-(4-fluoro-benzyl)-N″-n-propyl-[1,3,5]triazine-2,4,6-triamine;
O-(4-Fluorophenyl)-N-(4-n-propylamino-6-prop-2-ynylamino-[1,3,5]triazin-2-yl)-hydroxylamine;
N-[4-(1,1-Dimethyl-prop-2-ynylamino)-6-n-propylamino-[1,3,5]triazin-2-yl]-O,N-dimethyl-hydroxylamine;
O,N-Dimethyl-N-(4-n-propylamino-6-but-2-ynylamino-[1,3,5]triazin-2-yl)-hydroxylamine;
O,N-Dimethyl-N-(6-n-propylamino-2-prop-2-ynylamino-pyrimidin-4-yl)-hydroxylamine;
O,N-Dimethyl-N-(2-n-propylamino-6-prop-2-ynylamino-pyrimidin-4-yl)-hydroxylamine;
O,N-Dimethyl-N-(4-methylamino-6-prop-2-ynylamino-[1,3,5]triazin-2-yl)-hydroxylamine;
O,N-Dimethyl-N-(4-ethylamino-6-prop-2-ynylamino-[1,3,5]triazin-2-yl)-hydroxylamine;
O,N-Dimethyl-N-(4-isopropylamino-6-prop-2-ynylamino-[1,3,5]triazin-2-yl)-hydroxylamine;
O,N-Dimethyl-N-(4-cyclopropylamino-6-prop-2-ynylamino-[1,3,5]triazin-2-yl)-ydroxylamine;
O,N-Dimethyl-N-(4-n-butylamino-6-prop-2-ynylamino-[1,3,5]triazin-2-yl)-hydroxylamine;
O,N-Dimethyl-N-(4-cyclobutylamino-6-prop-2-ynylamino-[1,3,5]triazin-2-yl)-hydroxylamine;
O,N-Dimethyl-N-(4-cyclopropylmethylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-cyclohexylamino-6-prop-2-ynylamino-[1,3,5]triazin-2-yl)-hydroxylamine;
O,N-Dimethyl-N-(4-benzylamino-6-prop-2-ynylamino-[1,3,5]triazin-2-yl)-hydroxylamine;
O,N-Dimethyl-N-[4-(1-methyl-prop-2-ynylamino)-6-n-propylamino-[1,3,5]triazin-2-yl]-hydroxylamine;
O,N-Dimethyl-N-(4-but-3-ynylamino-6-n-propylamino-[1,3,5]triazin-2-yl)-hydroxylamine;
N-But-3-ynyl-N′-methyl-N″-n-propyl-[1,3,5]triazine-2,4,6-triamine;
O-tert-Butyl-N-(4-n-propylamino-6-prop-2-ynylamino-[1,3,5]triazin-2-yl)-hydroxylamine;
O-Ethyl-N-methyl-N-(4-n-propylamino-6-prop-2-ynylamino-[1,3,5]triazin-2-yl)-hydroxylamine;
O-Ethyl-N-(4-n-propylamino-6-prop-2-ynylamino-[1,3,5]triazin-2-yl)-hydroxylamine;
O-Methyl-N-(4-n-propylamino-6-prop-2-ynylamino-[1,3,5]triazin-2-yl)-hydroxylamine;
N-Methyl-N-(4-n-propylamino-6-prop-2-ynylamino-[1,3,5]triazin-2-yl)-hydroxylamine;
N-(4-n-Propylamino-6-prop-2-ynylamino-[1,3,5]triazin-2-yl)-hydroxylamine;
O-(2-Methoxy-ethyl)-N-methyl-N-(4-n-propylamino-6-prop-2-ynylamino-[1,3,5]triazin-2-yl)-hydroxylamine;
N-Methyl-O-(4,4,5,5,5-pentafluoropentyl)-N-(4-n-propylamino-6-prop-2-ynylamino-[1,3,5]triazin-2-yl)-hydroxylamine;
N-(4-Fluorophenyl)-N′-propyl-N″-prop-2-ynyl-[1,3,5]triazine-2,4,6-triamine;
N-(3-Chloro-2-methyl-benzyl)-N′-n-propyl-N″-prop-2-ynyl-[1,3,5]triazine-2,4,6-triamine;
N-(3,4-Dichlorobenzyl)-N′-n-propyl-N″-prop-2-ynyl-[1,3,5]triazine-2,4,6-triamine;
O,N-Dimethyl-N-(2-prop-2-ynylamino-7H-pyrrolo[2,3-d]pyrimidin-4-yl)-hydroxylamine;
N-(4,6-Bis-n-propylamino-[1,3,5]triazin-2-yl)-O-methyl-N-prop-2-ynyl-hydroxylamine;
O-Methyl-N-(4-n-propylamino-6-prop-2-ynylamino-[1,3,5]triazin-2-yl)-N-prop-2-ynyl-hydroxylamine;
N-(4,6-Bis-n-propylamino-[1,3,5]triazin-2-yl)-N-methyl-O-prop-2-ynyl-hydroxylamine;
N-(4,6-Bis-n-propylamino-[1,3,5]triazin-2-yl)-O-prop-2-ynyl-hydroxylamine;
N-Methyl-N-(4-n-propylamino-6-prop-2-ynylamino-[1,3,5]triazin-2-yl)-O-prop-2-ynyl-hydroxylamine;
N-(4-n-Propylamino-6-prop-2-ynylamino-[1,3,5]triazin-2-yl)-O-prop-2-ynyl-hydroxylamine;
N-(4-Allylamino-6-prop-2-ynylamino-[1,3,5]triazin-2-yl)-O,N-dimethyl-hydroxylamine;
1-[4-(N-Methoxy-N-methyl-amino)-6-prop-2-ynylamino-[1,3,5]triazin-2-ylamino]-propan-2-ol;
3-[4-(N-Methoxy-N-methyl-amino)-6-prop-2-ynylamino-[1,3,5]triazin-2-ylamino]-propan-1-ol;
N-(4-Amino-6-prop-2-ynylamino-[1,3,5]triazin-2-yl)-O,N-dimethyl-hydroxylamine;
3-[4-(N-Methoxy-N-methylamino)-6-prop-2-ynylamino-[1,3,5]triazin-2-ylamino]-propionaldehyde;
3-[4-(N-Methoxy-N-methylamino)-6-prop-2-ynylamino-[1,3,5]triazin-2-ylamino]-propionic acid ethyl ester hydrochloride;
N-Propyl-N′-prop-2-ynyl-[1,3,5]triazine-2,4,6-triamine;
N-[4-(N′-Methoxy-N′-methyl-amino)-6-prop-2-ynylamino-[1,3,5]triazin-2-yl]-N-propyl acetamide;
N-[4-(N′-Methoxy-N′-methyl-amino)-6-prop-2-ynylamino-[1,3,5]triazin-2-yl]-N-propyl adamantylamide;
N-Ethyl-N′-methyl-N″-prop-2-ynyl-[1,3,5]triazine-2,4,6-triamine;
N-Cyclopropyl-N′-methyl-N″-prop-2-ynyl-[1,3,5]triazine-2,4,6-triamine;
N-Butyl-N′-methyl-N″-prop-2-ynyl-[1,3,5]triazine-2,4,6-triamine;
N-Cyclopropylmethyl-N′-methyl-N″-prop-2-ynyl-[1,3,5]triazine-2,4,6-triamine;
N-Methyl-N′-prop-2-ynyl-N″-(3,3,3-trifluoro-propyl)-[1,3,5]triazine-2,4,6-triamine;
N-Methyl-N′-(2,2,3,3,3-pentafluoro-propyl)-N″-prop-2-ynyl-[1,3,5]triazine-2,4,6-triamine;
N-(1-Ethyl-propyl)-N′-methyl-N″-prop-2-ynyl-[1,3,5]triazine-2,4,6-triamine;
N,N-Dimethyl-N′-propyl-N″-prop-2-ynyl-[1,3,5]triazine-2,4,6-triamine;
N,N-Ethyl-methyl-N′-propyl-N″-prop-2-ynyl-[1,3,5]triazine-2,4,6-triamine;
N-Ethyl-N′-propyl-N″-prop-2-ynyl-[1,3,5]triazine-2,4,6-triamine;
N-Propyl-N′-propyl-N″-prop-2-ynyl-[1,3,5]triazine-2,4,6-triamine;
N-Cyclopropyl-N′-propyl-N″-prop-2-ynyl-[1,3,5]triazine-2,4,6-triamine;
N-Isopropyl-N′-propyl-N″-prop-2-ynyl-[1,3,5]triazine-2,4,6-triamine;
N-Butyl-N′-propyl-N″-prop-2-ynyl-[1,3,5]triazine-2,4,6-triamine;
N-Cyclopropylmethyl-N′-propyl-N″-prop-2-ynyl-[1,3,5]triazine-2,4,6-triamine; a salt thereof, and any combinations thereof.

In certain embodiments, the compound is selected from the group consisting of O,N-dimethyl-N-[4-(n-propylamino)-6-(prop-2-ynylamino-[1,3,5]triazin-2-yl]-hydroxylamine; N-methyl-N′-n-propyl-N″-prop-2-ynyl-[1,3,5]triazine-2,4,6-triamine; a salt thereof; and any combinations thereof.

In certain embodiments of the pharmaceutical composition, the stimulant is provided at a therapeutic dose which is sufficient to restore the respiratory rhythm of the patient.

In certain embodiments of the pharmaceutical composition, the stimulant is provided at a therapeutic dose which does not induce hyperventilation or substantial hyperventilation in the patient.

In certain embodiments, the respiratory stimulant is administered through an autoinjector containing a fixed dose or in combination of a fixed dose with other agents.

In a particular embodiment, the agent(s) are contained in a glass or plastic container.

In a particular embodiment, the composition is pre-mixed.

In a particular embodiment, the agent(s) are contained in a bifurcated applicator.

In a particular embodiment, one or both agents are lyophilized.

In certain embodiment, the respiratory stimulant is administered directly to the lung, e.g., by intratracheal instillation or intratracheal inhalation (e.g., with an endotracheal tube). In certain embodiments, the pulmonary is with a metered dose inhaler, nebulizer, soft mist inhaler, a high efficiency nebulizer, or ultrasonic nebulizer or dry powder inhaler.

In certain embodiments, the respiratory stimulant (optionally with additional agents such as bronchodilators) is administered with a continuous positive airway pressure (CPAP) machine, a bilevel positive airway pressure machine (BiPAP) or a ventilator.

In other embodiments, the respiratory stimulant can be a compound that acts on a steroid receptor mediated mechanism such as progesterone, a carotid body stimulant such as almitrine bismesylate, a carbonic anhydrase inhibitor such as acetazolamide, xanthines such as caffeine, theophylline, reflex stimulants such as ammonia and alcohol vapors, modafinil, and prethcamide.

The respiratory stimulant can include at least one compound of formula (I):

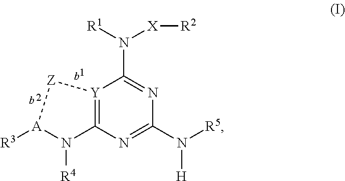

wherein:
R¹ and R² are independently H, alkyl, substituted alkyl, cycloalkyl, substituted cycloalkyl, alkenyl, substituted alkenyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, cycloalkyl, substituted cycloalkyl, alkenyl, substituted alkenyl, NR¹R², C(O)OR¹, acyl, or aryl;
R⁴ is H, alkyl, or substituted alkyl;
R⁵ is H, alkyl, substituted alkyl, cycloalkyl, substituted cycloalkyl, alkenyl, substituted alkenyl, —OR¹, —NR¹R², C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C; or a salt thereof.

In one embodiment, R³ is H, alkyl, substituted alkyl, cycloalkyl, substituted cycloalkyl, alkenyl, or substituted alkenyl. In another embodiment, R⁵ is H, alkyl, substituted alkyl, cycloalkyl, substituted cycloalkyl, alkenyl, substituted alkenyl, or acyl.

In one embodiment, the at least one compound of formula (I) is selected from the group consisting of: (i) Y is N, bond b¹ is nil, Z is H, bond b² is a single bond, A is CH, and the at least one compound is a compound of formula (II-a) or a salt thereof:

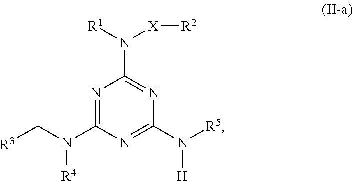

and
(ii) Y is N, bond b¹ is nil, Z is nil, bond b² is nil, and A is a bond, and the compound of the invention is a 1,3,5-triazine of formula (II-b) or a salt thereof:

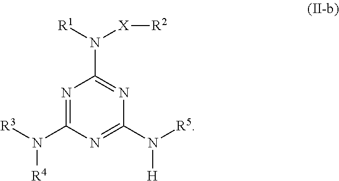

In one embodiment, the at least one compound of formula (I) is selected from the group consisting of: (i) Y is CR⁶, bond b¹ is nil, Z is H, bond b² is a single bond, A is CH, and the at least one compound is a compound of formula (III-a) or a salt thereof:

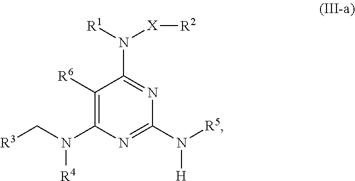

and
(ii) Y is CR⁶, bond b¹ is nil, Z is nil, bond b² is nil, and A is a bond, and the compound of the invention is a pyrimidine of formula (III-b) or a salt thereof:

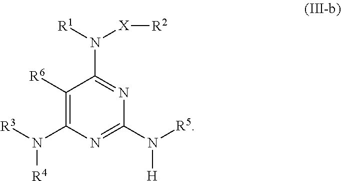

In one embodiment, Y is C, bond b¹ is a single bond, Z is CH₂, bond b² is a single bond, A is CH, and said at least one compound is a compound of formula (IV) or a salt thereof:

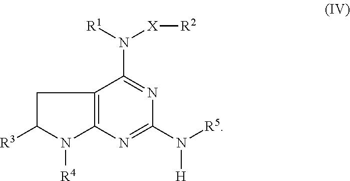

In one embodiment, Y is C, bond b¹ is a single bond, Z is CH, bond b² is a double bond, A is C, and said at least one compound is a compound of formula (V) or a salt thereof:

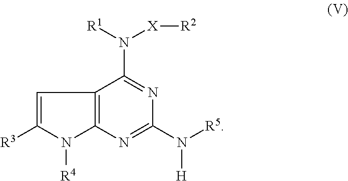

In one embodiment, the at least one compound is selected from the group consisting of: N-(4,6-Bis-methylamino-[1,3,5]triazin-2-yl)-N,O-dimethyl-hydroxylamine (XX), N-(4,6-Bis-ethylamino-[1,3,5]triazin-2-yl)-N,O-dimethyl-hydroxylamine (XXII), N-(4-Cyclopropylmethylamino)-N-(6-n-propylamino) [1,3,5]triazin-2-yl)-N,O-dimethyl-hydroxylamine (XXV), N-(4-Ethylamino)-N-(6-n-propylamino)-[1,3,5]triazin-2-yl)-N,O-dimethyl-hydroxylamine (XXVII), N-(Bis-4,6-(2-methylpropylamino)) [1,3,5]triazin-2-yl)-N,O-dimethyl-hydroxylamine (XXIX), N-(Bis-4,6-(2,2-dimethylpropylamino)) [1,3,5]triazin-2-yl)-O,N-dimethyl-hydroxylamine (XXXI), 4,6-Bis-N-cyclopropylamino-[1,3,5]triazin-2-yl)-N,O-dimethyl-hydroxylamine hydrochloride (XXXIII), N-(4,6-Bis-n-propylamino-[1,3,5]triazin-2-yl)-O,N-dimethyl-hydroxylamine (XXXV), N-(4-(Methoxy(methyl)amino)-6-(propylamino)-1,3,5-triazin-2-yl)propionamide (XL), N-(4,6-Bis-propylamino-[1,3,5]triazin-2-yl)-O-methyl-hydroxylamine (XLI), O-Allyl-N-(4,6-bis-propylamino-[1,3,5]triazin-2-yl)-hydroxylamine (XLIII), N-(4,6-Bis-propylamino-[1,3,5]triazin-2-yl)-hydroxylamine (XLV), 6-(Methoxy(methyl)amino)-N2-propyl-1,3,5-triazine-2,4-diamine (XLVII), N-(4,6-Bis-propylamino-[1,3,5]triazin-2-yl)-N-methyl-hydroxylamine (XLVIII), O-Benzyl-N-(4,6-bis-propylamino-[1,3,5]triazin-2-yl)-N-methyl-hydroxylamine (LIII), N-(4,6-Bis-propylamino-[1,3,5]triazin-2-yl)-N-isopropyl-hydroxylamine (LV), 6-[1,2]Oxazinan-2-yl-N,N′-dipropyl-[1,3,5]triazine-2,4-diamine (LVII), N-(4,6-Bis-propylamino-[1,3,5]triazin-2-yl)-O-isopropyl-N-methyl-hydroxylamine (LXIV), 0-Benzyl-N-(4,6-bis-propylamino-[1,3,5]triazin-2-yl)-N-ethyl-hydroxylamine (LXVIII), N-(4,6-Bis-propylamino-[1,3,5]triazin-2-yl)-O-isopropyl-hydroxylamine (LXX), 6-((Benzyloxy)(isopropyl)amino)-N2,N4-dipropyl-1,3,5-triazine-2,4-diamine (LXXII), N-(4,6-Bis-propylamino-[1,3,5]triazin-2-yl)-N-ethyl-O-isopropyl-hydroxylamine (LXXVI), N-(4,6-Bis-propylamino-[1,3,5]triazin-2-yl)-O-isobutyl-N-methyl-hydroxylamine (LXXXII), 6-(Methyl(thiophen-2-ylmethoxy)amino)-N2,N4-dipropyl-1,3,5-triazine-2,4-diamine (LXXXIV), N-(4,6-Bis-propylamino-[1,3,5]triazin-2-yl)-O-cyclopropylmethyl-N-methyl-hydroxylamine (XCI), N-(4,6-Bis-propylamino-[1,3,5]triazin-2-yl)-O-ethyl-N-methyl-hydroxylamine (XCVI), N-(4,6-Bis-propylamino-[1,3,5]triazin-2-yl)-O-(2,2-difluoro-ethyl)-hydroxylamine (C), 4-N-(2-Dimethylaminoethyl)amino-6-N-(n-propyl)amino-[1,3,5]triazin-2-yl)-N,O-dimethyl-hydroxylamine (CIII), 4-N-(3-(1-N-Methylimidazol-2-yl)-propyl)-amino-6-N-(n-propyl)amino-[1,3,5]triazin-2-yl)-N,O-dimethyl-hydroxylamine (CV), 4-N-(1-N-Methylimidazol-2-yl)-methylamino-6-N-(n-propyl)amino-[1,3,5]triazin-2-yl)-O,N-dimethyl-hydroxylamine (CVII), 4,6-Bis-(N-(2-dimethylaminoethyl)amino)-[1,3,5]triazin-2-yl)-N,O-dimethyl-hydroxylamine (CIX), 4,6-Bis-(N-(pyridin-4-ylmethyl)amino)-[1,3,5]triazin-2-yl)-N,O-dimethyl-hydroxylamine (CXI), 4,6-Bis-[N-(3-methoxy-n-propyl)amino]-[1,3,5]triazin-2-yl)-N,O-dimethyl-hydroxylamine (CXIII), 4,6-Bis-[N-(tetrahydropyran-4-ylmethyl)amino]-[1,3,5]triazin-2-yl)-N,O-dimethyl-hydroxylamine (CXV), N-(5,8,11-Trioxa-2,14,16,18,19-pentaazabicyclo[13.3.1]-nonadeca-1(18),15(19),16(17)-trien-17-yl)-N,O-dimethylhydroxylamine (CXVII), N-(4,6-Bis-propylamino-[1,3,5]triazin-2-yl)-N′,N′-dimethyihydrazine (XLVI), N-(4,6-Bis-propylamino-[1,3,5]triazin-2-yl)-N-methyl-N′-methyihydrazine (XLIX), a salt thereof and mixtures thereof. In another embodiment, the salt is hydrogen sulfate or hydrochloride.

In one embodiment, the at least one compound is 2,6-bis-(N-n-propylamino)-[1,3]pyrimidin-4-yl)-N,O-dimethyl-hydroxylamine N-(4-(Methoxy(methyl)amino)-6-(propylamino)-1,3,5-triazin-2-yl)propionamide or a salt thereof. In another embodiment, the salt is hydrogen sulfate or hydrochloride.

In one embodiment, the at least one compound is N-(4-(Methoxy(methyl)amino)-6-(propylamino)-1,3,5-triazin-2-yl)propionamide or a salt thereof. In another embodiment, the salt is hydrogen sulfate or hydrochloride.

In one embodiment, the at least one compound is selected from the group consisting of: 2-(n-Propyl)amino-4-(i-propylamino-7-methyl-pyrrolidino[2,3-d]pyrimidine (CXXVI), 2-(n-Propyl)amino-4-dimethylamino-7-methyl-pyrrolidino[2,3-d]pyrimidine (CXXVIII), 2-(n-Propyl)amino-4-methylamino-7-methyl-pyrrolidino[2,3-d]pyrimidine (CXXXI), 2-(n-Propyl)amino-4-(i-propyl)amino-7-i-propyl-pyrrolidino[2,3-d]pyrimidine (CXXXVI), 2,4-Bis-(n-propyl)amino-7H-pyrrolidino[2,3-d]pyrimidine (CXLIX), 2-(n-Propyl)amino-4-(4-hydroxypiperidin-1-yl)-7-methyl-pyrrolidino[2,3-d]pyrimidine (CLII), 8-(7-Methyl-2-(propylamino)-pyrrolidino[2,3-d]pyrimidin-4-yl)-8-azabicyclo[3.2.1]octan-3-ol (CLV), a salt thereof and mixtures thereof. In another embodiment, the salt is hydrogen sulfate or hydrochloride.

In one embodiment, the at least one compound is selected from the group consisting of: N-(2-Propylamino-7H-pyrrolo[2,3d]pyrimidin-4-yl)-O,N-dimethyl-hydroxylamine (CXLI), N-(2-(Propen-2-yl)amino-7-methyl-pyrrolo[2,3d]pyrimidin-4-yl)-N,O-dimethyl-hydroxylamine (CLVIII), N-(2-(Propen-2-yl)amino-7-methyl-pyrrolo[2,3d]pyrimidin-4-yl)-O-methyl-hydroxylamine (CLX), N-(2-n-Propylamino-7-methyl-pyrrolo[2,3d]pyrimidin-4-yl)-O,N-dimethyl-hydroxylamine (CLXII), N-(2-n-Propylamino-7-methyl-pyrrolo[2,3d]pyrimidin-4-yl)-O-methyl-hydroxylamine (CLXIV), N-(2-n-Propylamino-7-methyl-pyrrolo[2,3d]pyrimidin-4-yl)-hydrazine (CLXVI), N-Methyl-N-(2-n-propylamino-7-methyl-pyrrolo[2,3d]pyrimidin-4-yl)-hydrazine (CLXVIII), N,N-dimethyl-N′-(2-n-propylamino-7-methyl-pyrrolo[2,3d]pyrimidin-4-yl)-hydrazine (CLXX), a salt thereof and mixtures thereof. In another embodiment, the salt is hydrogen sulfate or hydrochloride.

In one embodiment of a parenteral formulation, the respiratory stimulant is doxapram or a pharmaceutically acceptable salt thereof such as the hydrochloride salt. The doxapram can be formulated as a parenteral product in an amount equivalent to 1 mg base/mL to about 50 mg/mL such as 10 mg/mL, 20 mg/mL or 30 mg/mL.

In certain embodiments, the respiratory stimulant can be administered with a bronchodilator. Suitable brochodilators include short-acting beta2 agonists, long-acting beta2 agonists, muscarinic agents, methylxanthines, short-acting anticholinergic agents, and combinations thereof.

Suitable short-acting beta2 agonists include albuterol, epinephrine, pirbuterol, levalbuterol, metaproteronol, pirbuterol, pharmaceutically acceptable salts thereof and combinations thereof.

Suitable long-acting beta2 agonists include salmeterol, formoterol and isomers (e.g. arformoterol), clenbuterol, tulobuterol, vilanterol, indacaterol, carmoterol, isoproterenol, procaterol, bambuterol, milveterol, olodaterol, pharmaceutically acceptable salts thereof and combinations thereof.

Suitable muscarinic agents include tiotroprium, trospium, glycopyrrolate, aclidinium, ipratropium, oxitropium, pharmaceutically acceptable salts thereof and combinations thereof.

Suitable methylxanthines include aminophylline, ephedrine, theophylline, oxtriphylline, pharmaceutically acceptable salts thereof and combinations thereof.

In one embodiment, the composition further comprises at least one pharmaceutically acceptable carrier.

In one embodiment, the compositions are formulated using one or more pharmaceutically acceptable excipients or carriers. In one embodiment, the pharmaceutical compositions. Pharmaceutically acceptable carriers, which are useful, include, but are not limited to, glycerol, water, saline, ethanol and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In certain embodiments, there can be the inclusion of isotonic agents, for example, sugars, sodium chloride, or polyalcohols such as mannitol and sorbitol, in the composition.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like.

The pharmacologically acceptable salts of any of the active agents utilized in the methods and compositions of the present disclosure can be, for example, an inorganic salt such as a hydrochloride, a sulfate, a phosphate or a hydrobromide; or an organic salt such as an oxalate, a malonate, a citrate, a fumarate, a lactate, a malate, a succinate, a tartrate, an acetate, a trifluoroacetate, a maleate, a gluconate, a benzoate, a salicylate, a xinafoate, a pamoate, an ascorbate, an adipate, a methanesulfonate, a p-toluenesulfonate or a cinnamate. These salts may be present in the form of a hydrate, a solvate or a crystalline polymorph.

In certain embodiments, the methods of the present invention are initiated when the patient has a blood oxyhemoglobin saturation level (e.g., SPO2 measurement) of less than about 90, less than about 80, less than about 75, less than about 60, less than about 50, less than about 40, less than about 30, less than about 20 or less than about 10. In other embodiments, the methods of the present invention raise the SPO2 level at least about 5%, at least about 10%, at least about 20%. At least about 35%, at least about 50%, at least about 65%, at least about 75%, at least about 85%, at least about 95%, at least about 100%, at least about 150%, at least about 200%, at least about 250%, at least about 350%, at least about 400% or at least about 500%.

In certain embodiments, the methods of the present invention are initiated when the patient has an arterial blood gas measurement for oxygen tension of less than about 80 mm hg, less than about 75 mm hg, less than about 60 mm hg, less than about 50 mm hg, less than about 40 mm hg, less than about 30 mm hg, less than about 20 mm hg or less than about 10 mm hg. In other embodiments, the methods of the present invention raise the arterial blood gas oxygen tension at least about 5%, at least about 10%, at least about 20%. At least about 35%, at least about 50%, at least about 65%, at least about 75%, at least about 85%, at least about 95%, at least about 100%, at least about 150%, at least about 200%, at least about 250%, at least about 350%, at least about 400% or at least about 500%.

The methods of the present invention can be initiated on a patient who is symptomatic of hypoxemia or who is asymptomatic of hypoxemia. In certain embodiments, the methods of the present invention are initiated on a patient who is experiencing one or more symptoms of hypoxemia selected from shortness of breath, chest pain, confusion, headache, rapid heartbeat and cyanosis. In certain embodiments, the methods of the present invention are initiated on a patient who is not experiencing any hypoxemia symptoms such as shortness of breath, chest pain, confusion, headache, rapid heartbeat and cyanosis but the patient has a blood oxyhemoglobin saturation level (e.g., SPO2 measurement; less than 90% etc.) or arterial blood gas measurement of oxygen tension (e.g. less than 80 mm hg etc.) that is as disclosed above.

The arterial blood gas measurement of oxygen tension can be performed by measuring oxygen in blood draw from an artery rather than a vein as blood in the arteries are oxygenated. Typically, the wrist arteries are used as they are more easily felt than other arteries of the body.

SPO2 is measured with a pulse oximeter (pulse ox) which is a noninvasive device that estimates the amount of oxygen in blood by sending infrared light into capillaries, e.g., in the finger, toe, or earlobe. The device then measures how much light is reflected off the gases and indicated the percentage saturation of blood hemoglobin with oxygen (SPO2).

In certain embodiments, the methods of the present invention are initiated when the patient has an arterial blood gas measurement for carbon dioxide tension, or an estimate of blood carbon dioxide tension through the use of a non-invasive measure such as end tidal CO2 (ETCO2), of greater than about 45 mm hg, greater than about 50 mm hg, greater than about 55 mm hg, greater than about 60 mm hg, greater than about 65 mm hg, greater than about 70 mm hg, greater than about 75 mm hg or greater than about 80 mm hg. In other embodiments, the methods of the present invention decrease the arterial blood gas carbon dioxide tension at least about 5%, at least about 10%, at least about 20%, at least about 35%, at least about 50%, at least about 65%, at least about 75%, at least about 85%, at least about 95%, at least about 100%.

The methods of the present invention can be initiated on a patient who is symptomatic of hypercapnia or who is asymptomatic of hypercapnia. In certain embodiments, the methods of the present invention are initiated on a patient who is experiencing one or more symptoms of hypercapnia selected from shortness of breath, confusion, headache and rapid heartbeat. In certain embodiments, the methods of the present invention are initiated on a patient who is not experiencing any hypercapnic symptoms such as shortness of breath, confusion, headache and rapid heartbeat but the patient has an arterial blood gas measurement, or non-invasively measured estimate (e.g., ETCO2), of carbon dioxide tension (e.g. greater than 45 mm hg etc.) that is discussed above.

1. A method of treating respiratory depression in a patient after discontinuation of ventilator therapy comprising administering a respiratory stimulant to a patient in need thereof.

2. The method of claim 1, wherein the ventilator therapy is to treat respiratory depression in a patient experiencing a bacterial or viral infection.

3. The method of claim 1, further comprising administering hydroxychloroquine to the patient.

4. The method of claim 1, further comprising administering an antibiotic to the patient.

5. The method of claim 4, wherein the antibiotic is azithromycin.

6. The method of claim 1, further comprising administering an antiviral to the patient.

7. The method of claim 6, wherein the antiviral is amantadine.

8. The method of claim 1, wherein the respiratory depression after discontinuation of ventilator treatment is caused by demyelination, atelectasis, or a combination thereof.

9. The method claim 1, wherein the ventilator therapy is to treat respiratory depression caused by administration of an active agent.

10. The method of claim 9, wherein the active agent is to treat pain, cough or constipation.

11. The method of claim 10, wherein the agent is an opioid analgesic.

12. The method of claim 1, wherein the respiratory stimulant triggers ventilation in the lungs.

13. The method of claim 12, wherein the wherein the respiratory stimulant triggers ventilation in the lungs through a physiological mechanistic process.

14. The method of claim 13, wherein the mechanistic process is through the carotid bodies.

15. The method of claim 1, wherein the respiratory stimulant is selected from the group consisting of Doxapram, Almitrine, ENA-001, and a pharmaceutically-acceptable salt thereof.

16. The method of claim 1, wherein the respiratory stimulant is at least one compound of formula (I): 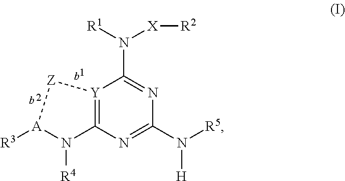
wherein:
R¹ and R² are independently H, alkyl, substituted alkyl, cycloalkyl, substituted cycloalkyl, alkenyl, substituted alkenyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, cycloalkyl, substituted cycloalkyl, alkenyl, substituted alkenyl, —NR¹R², —C(O)OR¹, acyl, or aryl;
R⁴ is H, alkyl, or substituted alkyl;
R⁵ is H, alkyl, substituted alkyl, cycloalkyl, substituted cycloalkyl, alkenyl, substituted alkenyl, —OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and, Y is N, CR⁶ or C; wherein:
if Y is N or CR⁶, then bond b¹ is nil and: (i) Z is H, bond b² is a single bond, and A is CH;
or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C;
or a salt thereof.

17. The method of claim 1, wherein the active agents are administered simultaneously or sequentially.

18. The method of claim 1, wherein at least two active agents are administered sequentially.

19. The method of claim 1, wherein the active agents are administered by the same route of administration.

20-32. (canceled)

33. A pharmaceutical composition comprising an effective amount of a respiratory stimulant to treat respiratory depression in a patient after discontinuation of ventilator therapy.

34-50. (canceled)
